(19) United States
(12) Patent Application Publication
(10) Pub. No.: US 2025/0384998 A1
(43) Pub. Date: Dec. 18, 2025

(54) MACHINE LEARNING TO SELECT TRANSMISSION TIMING

(71) Applicant: ResMed Digital Health, Inc., San Diego, CA (US)

(72) Inventors: Sakeena DE SOUZA, Sydney (AU); Jiaming CHEN, San Diego, CA (US); Nathan BARTLETT, Sydney (AU); Gregory GANCARZ, San Diego, CA (US); Alexis ISABELLE, Boxford, MA (US); Randall KELLEY, Natick, MA (US); Anish DALAL, Hampton Bays, NY (US); Sourav Raj DEY, San Francisco, CA (US)

(21) Appl. No.: 18/879,677

(22) PCT Filed: Jun. 28, 2023
(86) PCT No.: PCT/US2023/069293

§ 371 Date: Dec. 27, 2024

Related U.S. Application Data
(60) Provisional application No. 63/367,423, filed on Jun. 30, 2022.

(51) Int. Cl. G16H 40/60; G06N 20/00; G16H 10/60; G16H 20/40

(52) U.S. Cl. CPC G16H 40/60; G06N 20/00; G16H 10/60; G16H 20/40

(57) ABSTRACT

Techniques for improved machine learning are provided. A notification to be provided to a user engaged in a therapeutic treatment is identified, and a set of user characteristics associated with the user is determined. A target time to provide the notification to the user is identified by processing the set of user characteristics using a machine learning model, and the notification is transmitted to the user at the target time.

### CROSS-REFERENCE TO RELATED APPLICATIONS

This application is a 371 national phase application of PCT Application No. PCT/US2023/069293, filed Jun. 28, 2023, which claims benefit of co-pending U.S. provisional patent application Ser. No. 63/367,423 filed Jun. 30, 2022, the entire contents of which are incorporated herein by reference in its entirety.

### INTRODUCTION

Embodiments of the present disclosure relate to machine learning. More specifically, embodiments of the present disclosure relate to using machine learning to drive improved transmission timing.

In a wide variety of systems and environments, content or communications are frequently provided to clients from centralized systems. For example, a first entity (e.g., a business, a school, a governmental entity, and the like) may distribute content to users for a variety of purposes including providing information or guidance, offering services, providing reminders, and the like. In many conventional systems, various aspects of the content transmission are either fixed (e.g., the same for all users) or generally random (e.g., varying from user-to-user without reason). As a result, the content transmissions often fail to serve the underlying purpose (such as ensuring that users are informed of relevant information) well.

Systems and techniques to improve such content transmission are needed.

### SUMMARY

According to one embodiment presented in this disclosure, a method is provided. The method includes: accessing a plurality of notification records; determining, for at least a first notification record of the plurality of notification records: a first time when a notification was provided to a user; a second time when the notification was opened by the user, and a set of user characteristics associated with the user; and training a machine learning model, based on the plurality of notification records, to predict interaction probability of future notifications.

According to one embodiment presented in this disclosure, a method is provided. The method includes: identifying a notification to be provided to a user engaged in a therapeutic treatment; determining a set of user characteristics associated with the user; identifying a target time to provide the notification to the user by processing the set of user characteristics using a machine learning model; and transmitting the notification to the user at the target time.

Other embodiments provide processing systems configured to perform the aforementioned methods as well as those described herein; non-transitory, computer-readable media comprising instructions that, when executed by one or more processors of a processing system, cause the processing system to perform the aforementioned methods as well as those described herein; a computer program product embodied on a computer readable storage medium comprising code for performing the aforementioned methods as well as those further described herein; and a processing system comprising means for performing the aforementioned methods as well as those further described herein.

The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 depicts an example environment for using machine learning to improve content transmissions.

FIG. 2 depicts an example workflow for using and refining machine learning models to improve content transmissions.

FIG. 3 is a flow diagram depicting an example method for training machine learning models to improve content transmission timing.

FIG. 4 is a flow diagram depicting an example method for improving content transmission timing using trained machine learning models.

FIG. 5 is a flow diagram depicting an example method for refining content transmission machine learning models.

FIG. 6 is a flow diagram depicting an example method for training a machine learning model to predict the probability of user interaction with future notifications.

FIG. 7 is a flow diagram depicting an example method for using a machine learning model to transmit notifications at a predicted optimal time.

FIG. 8 depicts an example computing device configured to perform various aspects of the present disclosure.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for improved machine learning to improve content provisioning or transmission. In some embodiments, machine learning is used to predict an optimal or ideal time to transmit or otherwise provide content based on the individual characteristics of each specific user or client (e.g., of an application or mailing list) that receives content. Providing content in accordance with these predictions can significantly increase the probability that the content will be interacted with by the intended recipient, such as being received, reviewed, and/or acted upon by a recipient.

In some aspects of the present disclosure, communications and/or notifications related to therapeutic treatments are provided as example content that can be customized using machine learning. For example, users of continuous positive airway pressure (CPAP), or bi-level positive airway pressure (BiPAP), and/or automatic positive airway pressure (APAP) devices may be provided content such as coaching or other information in order to improve their experience and use of the therapy (e.g., tips to reduce mask leakage, reminders to use their devices, and the like). Notwithstanding these and other examples, embodiments of the present disclosure are readily applicable to any other content provisioning or transmission medical purposes (e.g., providing updates or information related to various therapeutic or medication plans), other reminders or instructional content, and the like.

In some embodiments, user characteristics such as age, the length of time (e.g., the number of days, weeks, or months) they have used a medical device (e.g., a CPAP machine), the average duration of such usage (e.g., hours per day), the standard deviation of such usage, and the like can be used as input to one or more trained machine learning models in order to generate a predicted optimal timing to deliver or provide content to the user (e.g., via a notification or other communication). In some embodiments, the model provides a recommendation corresponding to the predicted optimal hour of the day to send the communication (e.g., 10:00 in the morning or 3:00 in the afternoon) based on the specific user characteristics. Though some examples herein describe prediction of an optimal hour, in some embodiments, the model can additionally or alternatively predict more specific times (e.g., a specific minute), or a less specific time, such as morning or evening. In at least one embodiment, the model can be used to determine an hour value (e.g., between zero and twenty-three) indicating the predicted ideal time to provide the content (e.g., where a value of ten indicates 10:00 in the morning, and a value of fifteen indicates 3:00 in the afternoon).

In some embodiments, the machine learning model operates by generating a probability that the user will open, view, or otherwise interact with the content within a defined maximum length of time from when it is provided or transmitted. That is, the model may receive the user characteristics and a proposed time as input, and generate an interaction probability indicating the likelihood that the user will interact with the content (e.g., open, view, etc.) within the time duration, assuming it is provided or transmitted at the indicated proposed time. By evaluating multiple alternative times sequentially or in parallel, the system can identify the optimal time (e.g., the time that resulted in the highest interaction probability). For example, the system may use an argmax operation on the set of probabilities (one for each alternative time) to find the time having the highest interaction probability. In some embodiments, rather than receiving a proposed time as input, the model may process the user characteristics and output one or more proposed times, each with a respective interaction probability.

In embodiments of the present disclosure, the term “interaction probability” may be used to generically refer to the probability or likelihood that a recipient user will interact with a provided notification, communication, or other content according to one or more triggering criteria within a defined window or duration of time (e.g., a defined number of hours). For example, the “interaction probability” may correspond to the likelihood that the user opens the content, views the content, selects or activates the content, follows one or more links included in the content, finishes viewing the content, responds to the content, and the like.

In some embodiments, the triggering event (e.g., opening the content, viewing the content, activating or otherwise interacting with the content, and the like) can be defined by a user or administrator prior to training the model. Additionally, in some embodiments, the desired maximum length of time can be similarly defined. In at least one embodiment, multiple such models may be trained for a variety of predictions. For example, a first model may be trained to predict the probability that the user will open an electronic mail (email) message within three hours, and a second model may be trained to predict the probability that the user will select a link or otherwise engage with the content of the email within six hours. In one such embodiment, the models may be used in concert to select an optimal target time based on a variety of desired triggering events. For example, one or more functions or algorithms (e.g., a softmax function) may be used to evaluate the probabilities generated by each model to generate an overall score indicating the predicted value or aggregate benefit of using the specific time to transmit the content. As one example, a given time may be selected because it has the highest probability of further engagement or interaction, even if the probability that the user will quickly open the message is lower than other times.

In some embodiments, the machine learning models can be trained offline and deployed for runtime use. For example, the system may collect and/or annotate prior communication records (also referred to as notification records, transmission records, provisioning records, and/or content records) to train the model. In some embodiments, the prior records may include information such as the characteristics of the recipient user, the time the communication was provided or transmitted (e.g., the time it was sent and/or the time it was delivered), the time the recipient opened the communication (or the time of some other triggering event), the time that elapsed between the provision time and triggering time, whether the elapsed time is within one or more defined threshold durations, and the like. Using this data, the system may train a model to predict whether the indicated triggering event will occur within the indicated threshold time based on the user characteristics and the time the communication is provided.

In an embodiment, after training, the model may be deployed for runtime use. For example, when new content is being prepared for transmission to a user, the system may use the trained machine learning model to generate an optimal or target provision time (e.g., by processing the user characteristics and one or more alternative times as input, and selecting the time having the highest probability). When the predicted optimal time arrives, the system can automatically transmit or otherwise provide the content.

In some embodiments, after the content is provided or transmitted, the system can monitor for one or more triggering events in order to create further training or refinement data. For example, the system may determine whether the user opened or otherwise interacted with the content within the defined duration, and generate a communication record for the event (e.g., indicating the user characteristics, the time the content was provided, and whether the user opened the content within the defined duration). These records can then be used to subsequently refine the model (either continuously or periodically). In some embodiments, the model can be updated centrally (e.g., on a cloud system or other training system) and distributed to other devices for use in runtime.

In embodiments, the present disclosure enables improved communications and content delivery (including over various wireless or wired networks) by targeting specific and optimal delivery or transmission times. As the models can improve the probability that the user adequately interacts with the content, embodiments of the present disclosure can reduce bandwidth consumption by avoiding repeated transmissions. Particularly at scale, a vast amount of computational and networking resources are consumed in sending such messages. This is a significant technological problem in modern systems. Using embodiments of the present disclosure, however, the user is more likely to actually receive or interact with the content, which reduces the probability that follow-up content (e.g., reminders) will be needed. This reduction in network transmissions reduces the burden on the communication systems and the network itself, thereby significantly improving the operations of the network and computing systems themselves.

### Example Environment for Using Machine Learning to Improve Content Transmissions

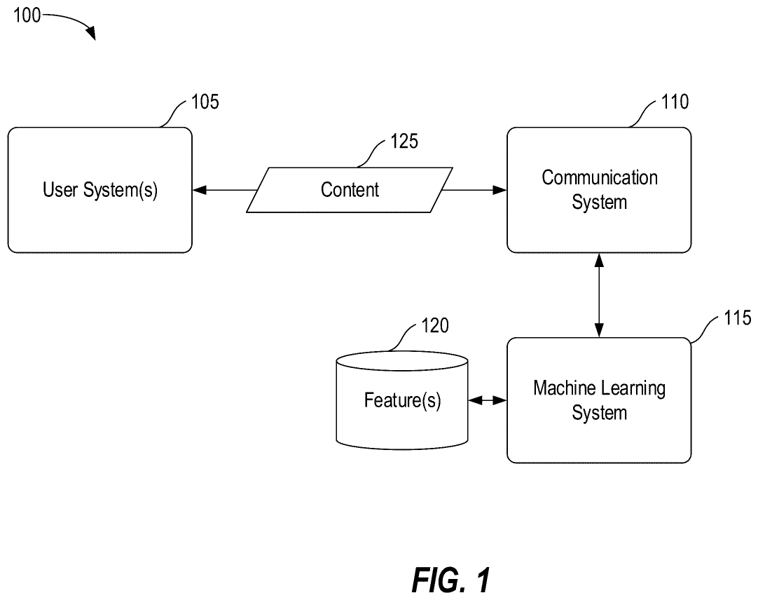

FIG. 1 depicts an example environment 100 for using machine learning to improve content transmissions.

In the illustrated environment 100, a user system 105 and a communication system 110 are communicably linked by one or more networks (not illustrated). The communication link between the user system(s) 105 and communication system 110 may generally include any suitable connection, including wireless communications, wired communications, or a combination of both wireless and wired communications. In at least one embodiment, the communication link includes the Internet. Although the illustrated example depicts a direct communication link between the user system 105 and communication system 110 for conceptual clarity, in embodiments, the content 125 may be provided via one or more intermediaries. For example, the content 125 may be provided as an email, instant message, text message, push message, or other communication that may pass through or ultimately reside on one or more other systems until the user system 105 receives or retrieves it. For example, the content 125 may be presented to a user of user system 105 within or as a part of an application resident on user system 105. As one example, the user system 105 may have an application related to an ongoing therapy (e.g., to control, record, or otherwise monitor CPAP usage), and the content 125 may be output via this application (e.g., as a push notification, or as an in-application message).

In an embodiment, the user system 105 generally corresponds to a computing system or device used by a user to receive, review, or otherwise interact with content 125. For example, the user system 105 may correspond to a laptop computer, a desktop computer, a smartphone, a tablet, a smart wearable (e.g., a smart watch), and the like. As discussed above, the content 125 can generally correspond to any communication or notification, including email, text messaging, phone calls, video and/or audio messages or calls, application-specific messages or notifications, and the like. Although a single user system 105 is depicted for conceptual clarity, in embodiments, there may be any number and variety of user systems used by any number of users. In some embodiments, the user system 105 is coupled with, linked to, or otherwise associated with other user devices (e.g., medical devices), such as a CPAP machine. In one such embodiment, the content 125 may relate to the use or control of such devices. For example, the content 125 may be a reminder relating to use of a medical device, instructions or suggestions for the device, and the like. In at least one embodiment, the user system 105 is, itself, a medical device (e.g., a smart CPAP machine).

In an embodiment, the communication system 110 generally corresponds to a computing system or device that manages, generates, and/or provides content 125 to users via user systems 105. In some embodiments, the communication system 110 may be used as part of one or more therapeutic treatments to engage with or otherwise provide content 125 to users engaging in the therapy. For example, the users may correspond to users of CPAP, BiPAP, and/or APAP machines, and the communication system 110 may be used or operated by a healthcare provider (e.g., the provider of the CPAP machines, doctors caring for the users, and the like) to provide engagement including coaching or encouragement, instructions, additional information, and the like. In some embodiments, the communication system 110 (or other systems) can monitor interactions and use of these related devices (e.g., determining whether the user actually uses the CPAP machine more consistently) in order to monitor whether the delivery, timing, and/or actual content of the content 125 was useful. Though a single communication system 110 is depicted for conceptual clarity, there may be multiple such systems.

As illustrated, the communication system 110 can interact with a machine learning system 115 to optimize the provisioning or transmission of the content 125. Although depicted as a discrete system for conceptual clarity, in some embodiments, the operations of the machine learning system 115 may be implemented within the communication system 110 itself. Additionally, in some embodiments, a discrete machine learning system 115 may be used to train the machine learning models, which can then be deployed on the communication system 110 for runtime use.

In some embodiments, the machine learning system 115 can be used to determine a target time (also referred to as a predicted time, a predicted target time, an optimal time, a predicted optimal time, an ideal time, a predicted ideal time, a best time, and/or a predicted best time) to provide content 125 based on characteristics of the recipient, as discussed in more detail below. For example, the communication system 110 may provide information such as the user characteristics, the content to be provided, and the like. In some embodiments, the communication system 110 can indicate the recipient, and the machine learning system 115 can retrieve the user characteristics (e.g., from the features 120). In some embodiments, the features 120 contains the relevant user data. In at least one embodiment, the features 120 can correspond to or receive the relevant information from one or more other databases, such as customer databases, application user databases, and the like. The machine learning system 115 can then use all or a subset of this data to predict the ideal transmission time, such as by processing one or more alternative times, alongside the user characteristics, as input to the model.

In some embodiments, the machine learning system 115 trains and/or refines the machine learning model(s) based on the provided content 125, as discussed in more detail below. For example, based on prior notifications or communications, the machine learning system 115 can refine the models to improve future predictions.

In the illustrated example, the machine learning system 115 uses a set of features 120 to define the machine learning models and/or process new input. The features 120 generally correspond to the data that is used as input to the model(s). In embodiments, the features 120 may be manually selected or defined (e.g., by an administrator), automatically curated or selected from a larger set of possible features (e.g., using one or more feature selection techniques), or a combination of the two. For example, one or more features may be manually selected by an architect of the model, and the machine learning system 115 may evaluate the features (e.g., by training a model that uses the features, or by using feature selection techniques) to identify which features are most salient and which are not useful (or are less useful than a threshold amount) in the prediction task.

In some embodiments, the features 120 can generally include aspects or characteristics of the recipient user. By way of example, if the machine learning system 115 is used to improve communications or notifications to CPAP, BiPAP, and/or APAPs users, the features 120 may include attributes such as the recipient's age (e.g., in years), the duration (e.g., the number of days) that the recipient has used the CPAP/BiPAP/APAP device or otherwise been involved in the therapy, the gender of the recipient, the average duration (e.g., number of hours) that the recipient uses the medical device per session (e.g., per day or per overnight period), a sleep apnea/sleep hypopnea (AHI) index of the recipient, which may include the diagnosis AHI measured when the user was diagnosed, and/or the residual AHI while the user is on the therapy using the device (e.g., the number of residual apnea events (cessation of breathing for a defined period, such as at least ten seconds) and/or hypopnea events (partial cessation of breathing for a defined period, such as at least ten seconds) that a user experiences per hour), a mean or average device usage per day (e.g., in hours) by the recipient over a defined number of days (e.g., the last three days), a standard deviation of the mean or average device usage per day over the defined window of days, a median or average amount of mask leakage of the device used by the recipient (e.g., the liters per second or per minute, averaged over a defined period of days), the standard deviation of such mask leakage over a defined window of time (e.g., the last three days), the phenotype of the recipient (e.g., a label representing the usage-group of the patient depending on their usage pattern of the device, such as “new,” “full-time,” “part-time,” or “stopped”), the compliance status of the recipient (e.g., “compliant,” “not-compliant,” “eligible,” or “unknown,” as determined by the number of observed nights of mask usage that meets or exceeds a defined amount, such as at least four hours usage of the device, over a period or window of days), the total number of positive airway pressure (PAP) therapy devices used by the user (e.g., whether they have one CPAP, one CPAP and one APAP, and the like), details on the handset platforms the user uses to access or view information relating to the therapy, such as types and/or numbers of handsets/devices (e.g., the number of smartphones of one brand, the number of smartphones of another brand, the number of web platforms the user uses, and the like), a user-provided (self-reported) diagnosis AHI, the type of sleep test that the user used to identify or diagnose their apnea (e.g., a polysomnography (PSG) or home sleep test (HST)), the type of mask that the user uses with their PAP machine(s) (e.g., nasal pillows, full-face, and the like), and/or the device name of the PAP device used by the user (e.g., the brand name and/or model).

In an embodiment, after the machine learning model is trained, the machine learning system 115 can indicate, to the communication system 110, the ideal time to transmit, deliver, otherwise provide the content 125. In some embodiments, the communication system 110 and/or machine learning system 115 can thereafter monitor the content 125, user system 105, or other information to determine whether any triggering events have occurred. For example, the machine learning system 115 may determine whether the recipient opened the content 125, the time of the opening, and the like. In an embodiment, this feedback data can be used to further refine or fine-tune the machine learning model(s), resulting in improved predictions, as discussed in more detail below.

### Example Workflow for Using and Refining Machine Learning Models to Improve Content Transmissions

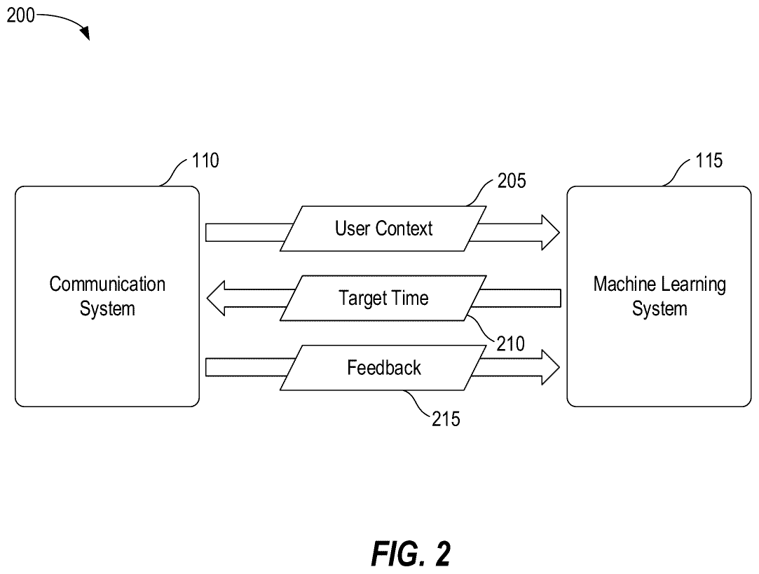

FIG. 2 depicts an example workflow 200 for using and refining machine learning models to improve content transmissions. In some embodiments, the workflow 200 is used after initial training of the machine learning model(s).

In the illustrated example, a communication system 110 is communicably coupled with the machine learning system 115, such as via the Internet. Although depicted as discrete systems for conceptual clarity, as discussed above, some or all of the operations of machine learning system 115 and the communication system 110 may be performed on a single system, or may be distributed across multiple systems. For example, the communication system 110 may instantiate a copy of the trained machine learning model(s) for local inferencing.

In order to better provide content to a specific recipient or user, in the illustrated workflow 200, the communication system 110 can transmit an indication of the user context 205 to the machine learning system 115. In an embodiment, the user context 205 can generally include information relating to, describing, or identifying the desired recipient(s) of content. For example, in some embodiments, the user context 205 indicates the user attributes or characteristics, such as their age, how long they have participated in a therapy program, and the like. In some embodiments, the user context 205 identifies the recipients (e.g., by name or by some other unique identifier), and the machine learning system 115 uses the identifier to access or retrieve the user attributes or characteristics of the indicated recipient(s).

As illustrated, based on the user context 205, the machine learning system 115 generates and returns a target time 210. As discussed above, the target time 210 may generate the target time 210 by processing one or more aspects of the communication/content, the intended recipient, and/or one or more alternative transmission times using one or more trained machine learning models. For example, the machine learning system 115 may process the recipient's characteristics (e.g., age, how long they have used a given medical device, and the like) and a proposed time (e.g., an hour of day when the content will be provided or transmitted) as input to the model. In such an embodiment, the model may output a value indicating the probability that the recipient user will open, interact with, or otherwise engage with the content within a defined window of time (e.g., within six hours).

In some embodiments, by processing multiple alternative times (along with the user characteristics) sequentially or in parallel, the machine learning system 115 can identify the time(s) having the highest probability that the user will open the content within the defined window. For example, suppose the machine learning system 115 processes the user characteristics and a proposed transmission time of noon (e.g., an hour value of 12) using the machine learning model, generating an interaction probability of 0.6 (indicating that there is a 60% chance the user will open the content within the defined window of time). Suppose further that the machine learning system 115 processes the user characteristics and a second proposed transmission time of 5:00 pm (e.g., an hour value of 17) using the machine learning model, generating an interaction probability of 0.72 (indicating that there is a 72% chance the user will open the content within the defined window of time). In an embodiment, the machine learning system 115 may return 5:00 pm as the target time 210 (assuming that no other proposed times exceed an interaction probability of 0.72).

In an embodiment, the communication system 110 can then wait until the indicated target time 210 arrives. Upon determining that the target time 210 has been reached, the communication system 110 can transmit or otherwise provide the content to the corresponding recipient user. As the specific attributes of each respective user may differ, the ideal target time 210 for each respective user can similarly differ. Thus, in an embodiment, the communication system 110 may transmit the same content to different users at different times (or may transmit custom-tailored content to each user), thereby improving the probability that each individual user will respond to the content appropriately or as desired.

In some embodiments, rather than a single target time 210, the machine learning system 115 can indicate interaction probabilities for multiple alternative times. For example, for each possible hour (which may include all twenty-four hours in a day, or may correspond to a subset, such as ordinary business hours), the machine learning system 115 may indicate a respective interaction probability. Similarly, in at least one embodiment, the machine learning system 115 may identify a preferred subset of alternative times by comparing interaction probabilities to one or more thresholds, and provide the preferred subset of alternatives (e.g., indicating any transmission times with an interaction probability that meets or exceeds a minimum threshold probability, such as 0.5).

In some embodiments, if the target time 210 has already passed (e.g., if the target time is noon but it is currently 3:00 in the afternoon), the communication system 110 can determine to transmit the content to the user on the following day (e.g., at noon on the subsequent day). In other embodiments, the communication system 110 may determine to immediately transmit the content (e.g., if it is preferable to provide the content today, rather than wait for a more ideal time of day tomorrow).

In at least one embodiment, if the machine learning system 115 indicates multiple alternative times and/or interaction probabilities for multiple times, but the highest-scored time has already passed, the communication system 110 can determine whether to transmit the content later on the current day (e.g., if a future time has a sufficiently-high interaction probability) or to wait until the ideal time on the following day.

As illustrated, after the content is provided, the communication system 110 (or another system) can evaluate the recipient user's engagement with it. For example, depending on the particular triggering event used by the system, the communication system 110 may determine when the user opens the content, views the content, responds to the content, and the like. In some embodiments, the communication system 110 can collect timing data for a variety of triggering events (e.g., determining not only when the user opened the content, but also when they closed it or finished viewing it, when they responded to it, when the accepted or approved it, and the like). In some embodiments, as discussed above, the communication system 110 can additionally or alternatively collect data from other devices or systems (e.g., CPAP machines), as discussed above. For example, the communication system 110 can determine whether the user used the device, adjusted settings on it, and the like after the content was provided.

In the depicted workflow 200, the communication system 110 can provide feedback 215 to the machine learning system 115 based on this evaluation or monitoring. For example, in one such embodiment, the communication system 110 can indicate the recipient user (and/or the user's characteristics or attributes) and the time that the content was actually provided to the user, along with timing information for one or more triggering events. In an embodiment, the timing information for the triggering events can include indicating the time(s) that the triggering event(s) occurred, how much time elapsed from when the content was provided to when the triggering event(s) occurred, whether the elapsed time exceeds a threshold maximum duration, and the like.

Using the feedback, the machine learning system 115 can refine or fine-tune the machine learning model(s). For example, the feedback 215 can be used as a new training exemplar, where the user attributes and the actual transmission time are provided as input to generate an interaction probability. This generated probability can then be compared against the ground-truth timing information provided in the feedback 215 in order to refine the model to make more accurate predictions. In some embodiments, this refining process can be performed continuously, periodically (e.g., weekly) based on batches of feedback 215, and the like.

### Example Method for Training Machine Learning Models to Improve Content Transmission Timing

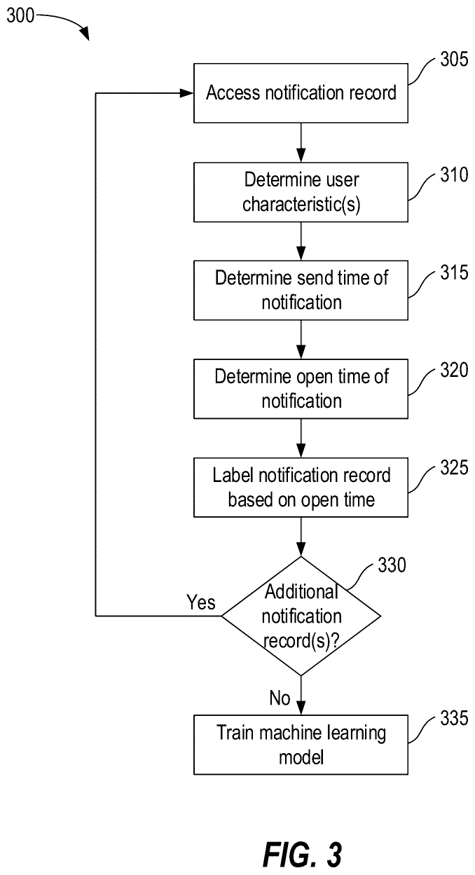

FIG. 3 is a flow diagram depicting an example method 300 for training machine learning models to improve content transmission timing. In some embodiments, the method 300 is performed by a machine learning system, such as machine learning system 115 of FIGS. 1 and 2.

The method 300 begins at block 305, where the machine learning system accesses a notification record. The notification record generally includes information relating to the provisioning of content to a recipient user at one or more prior times in the past. For example, the notification record may correspond to an email was transmitted to a user. In some embodiments, accessing the notification record corresponds to receiving or retrieving it from one or more other systems, such as the communication system 110 of FIGS. 1 and 2. In some embodiments, accessing the notification record includes accessing from a local storage or memory.

At block 310, the machine learning system determines the user characteristics of the recipient indicated in the notification record. For example, in some embodiments, the notification record itself indicates the relevant user characteristics (e.g., where the relevant characteristics are those that are used by the machine learning system). In another embodiment, the notification record may uniquely identify the user (e.g., via an identification number), and the machine learning system can determine or retrieve the user characteristics from one or more other sources using the identifier.

In some embodiments, determining the user characteristics can include one or more preprocessing steps to prepare the data for use in training the machine learning model(s). For example, in one embodiment, the machine learning system can determine whether any of the relevant data is missing. That is, the machine learning system can determine whether the user characteristics do not indicate a value for one or more of the features that are used by the system. If so, the machine learning system may use a variety of techniques to prepare the record for training.

In one embodiment, if the missing data corresponds to a categorical feature (e.g., a phenotype feature or a mask type feature), the machine learning system may identify the category that is the most common value for the missing data, among the available notification records. That is, if the currently-selected record has a missing value for a given categorical feature, the machine learning system may evaluate other notification records to determine the most-common category, and fill in the user characteristics of the current record using this category. For example, for a “phenotype” feature, the machine learning system may determine that “full-time” is the most common category, and add this data to the notification record.

In some embodiments, if the missing data corresponds to a continuous or numerical feature, the machine learning system can determine the average or median value reflected in one or more other notification records. That is, if the currently-selected record has a missing value for a given numerical feature, the machine learning system may evaluate other notification records to determine the median or average value for the feature, and fill in the user characteristics of the current record using this value. For example, for a “mask leak” feature, the machine learning system may determine the average value in the other records, and add this data to the notification record.

In some embodiments, determining the user characteristics can additionally or alternatively include encoding them to prepare them for training. For example, the machine learning system may use one-hot encodings for categorical features (e.g., where each categorical feature is associated with a corresponding input vector, each input vector has a length equal to the number of categories for the corresponding feature). In such an embodiment, encoding the categorical features can include setting the appropriate entry in the vector (e.g., the entry corresponding to the category indicated in the user data) to a non-zero value (e.g., to one) and setting all other entries to zero.

In at least one embodiment, when using one-hot encodings, in order to avoid or reduce collinearity, the last category can be omitted from processed training predictor columns. For example, if a given categorical feature has four categories, the system may omit the fourth category, as its value can be inferred by the first three (e.g., if the first three are zero, it can be inferred that the user belongs to the fourth category). This can reduce collinearity and/or computational expense.

At block 315, the machine learning system can determine the send time of the notification that corresponds to the selected notification record. In some embodiments, the send time may refer to the actual time that the content was sent or transmitted. In at least one embodiment, determining the send time can correspond to determining the nearest whole hour for when the content was provided. For example, if the content was actually provided at 3:15 pm, the machine learning system may determine the send time as 3:00 pm. Similarly, if the send time was 3:50 pm, the machine learning system may determine the send time as 4:00 pm.

At block 320, the machine learning system can similarly determine the open time of the notification. In embodiments, the open time may correspond to the time when the user triggered one or more events, such as opening the content, viewing the content, closing the content, responding to the content, and the like. In at least one embodiment, determining the open time can correspond to determining the nearest whole hour for when the content was opened, as discussed above. In some embodiments, determining the open time corresponds to determining the amount of time that elapsed between the send time and the open time.

In some embodiments, determining the send time and open time can additionally or alternatively include encoding them to prepare them for training. For example, the machine learning system may augment the send time and/or open time using cyclical features to account for the twenty-four hour daily cycle. In an embodiment, these cyclical features can allow the machine learning model(s) to consider the fact that a value of zero (e.g., corresponding to midnight) is close to a value of 23 (e.g., corresponding to a time of 11:00 pm), even though these values are distant from each other. In at least one embodiment, the machine learning system can generate the cyclical features by normalizing the send time (e.g., by multiplying the send time by 2π and dividing by 23. A first cyclical feature can then be generated by applying the sine function to the normalized value, and a second feature is created using the cosine function on the normalized value. In an embodiment, these cyclical features can be used as the send time feature when training the model.

At block 325, the machine learning system can label the selected notification record based on the determined open time. For example, in some embodiments, the machine learning system can label the notification record with a binary value indicating whether the user opened the content (or performed some other triggering action) within a defined maximum duration of time. In some embodiments, the machine learning system may additionally or alternative label the record to indicate the amount of time that elapsed until the open time. In some embodiments, if no open time (or other interaction time) is available (e.g., because the user didn't interact with or open the content), the machine learning system can label the notification record to indicate that the content was not opened (or was not opened within the designated duration of time).

At block 330, the machine learning system can determine whether there is at least one additional notification record that has not-yet been evaluated. If so, the method 300 returns to block 305. If not, the method 300 continues to block 335. Although the illustrated example depicts an iterative process (evaluating each notification record sequentially) for conceptual clarity, in some embodiments, some or all of the notification records may be evaluated in parallel, and/or they may be evaluated and labeled as they are created (e.g., when the open event occurs).

At block 335, the machine learning system trains one or more machine learning models based on the labeled notification records. In some embodiments, this may include refining the model individually using each notification record alone (e.g., using stochastic gradient descent for a neural network model). In some embodiments, the training includes refining the model using multiple records combined (e.g., using batch gradient descent for a neural network model). In some embodiments, training the machine learning model includes training or refining a random forest model with a binary target predicting the probability of the email being opened within the defined time duration.

Once the model is trained, it can be deployed for runtime inferencing use, where user characteristics are processed to identify or predict an optimal or ideal target time to transmit content to the specific user (e.g., on the machine learning system 115 of FIGS. 1 and 2).

### Example Method for Improving Content Transmission Timing Using Machine Learning

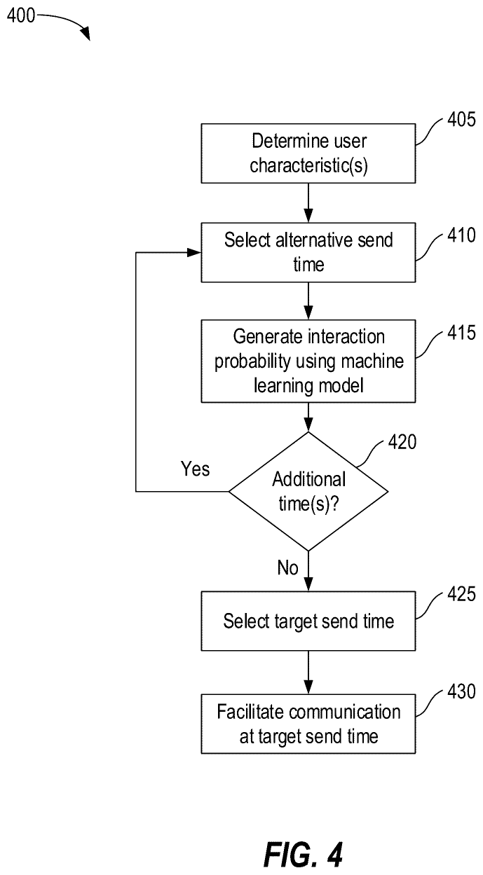

FIG. 4 is a flow diagram depicting an example method 400 for improving content transmission timing using trained machine learning models. In some embodiments, the method 400 is performed by a machine learning system, such as machine learning system 115 of FIGS. 1 and 2. In other embodiments, the method 400 is performed by a communication system, such as communication system 110 of FIGS. 1 and 2.

At block 405, the computing system determines a set of user characteristics for an intended or planned recipient of a notification, alert, or other communication. For example, as discussed above, the recipient may be a user of a medical device and/or a participant in a therapeutic regimen, such as a user of a CPAP, BiPAP, and/or APAP machine. In aspects, as discussed above, the user characteristics can generally include a variety of attributes or other information of the user, such as their age, the average length of time they use their medical device during a defined window of time (e.g., the average number of hours per day over the last three days), the number of days or weeks they have used the device, and the like.

In some embodiments, as discussed above, the computing system may additionally perform various preprocessing on the user characteristics. For example, the computing system may fill in or estimate any missing values based on the average or most-common value of the feature from a set of other users. In some embodiments, the computing system can encode some or all of the data (e.g., using one-hot encoding) to prepare it for processing with the machine learning model.

At block 410, the computing system selects a potential or alternative send time from a set of possible alternatives. For example, as discussed above, the computing system may be tasked with selecting or indicating the best hour of day for transmitting the content, based on the specific user characteristics. In some embodiments, the computing system selects from among a defined subset of alternative times (e.g., between 9:00 am and 5:00 pm). In at least one embodiment, the computing system can select from a set of alternatives that have not yet passed. That is, the computing system may determine the current time, and select alternatives that are equal to or greater than the current time (e.g., in the future on the same day).

In some embodiments, rather than selecting an hour (e.g., where the alternatives are delineated into hour-long increments), the computing system can select a more specific time, such as in thirty-minute increments, in fifteen-minute increments, and the like. In embodiments, the computing system may select the alternative time according to any criteria, as the computing system will evaluate all alternatives.

In some embodiments, the computing system can additionally perform preprocessing or encoding operations on the selected alternative send time. For example, as discussed above, the computing system may generate cyclical features for the selected time by normalizing the selected time and/or processing the selected time (or the normalized value) using a sine function and/or a cosine function. In one such aspect, two cyclical features are generated for the send time (e.g., one using sine and one using cosine), and these cyclical features are used as input to the model (rather than inputting the original time value).

At block 415, the computing system generates an interaction probability by processing the user characteristics and the alternative send time (or the encoded cyclical features) using a trained machine learning model (e.g., using a local model, or by requesting that another system, such as the machine learning system 115 of FIGS. 1 and 2 process the data). In an embodiment, as discussed above, the interaction probability indicates the likelihood that the recipient user will open (or otherwise interact or engage with) the content within a defined maximum duration from when it is provided. For example, the interaction probability may indicate the likelihood that the user will open an email containing the content within six hours of delivery. In an embodiment, the duration may be a predefined value used when training the model, as discussed above. By processing the user characteristics alongside the selected time, the computing system is able to predict how changing the send time affects the probability that the specific user will open (or otherwise engage with) the content in a timely manner.

At block 420, the computing system determines whether there is at least one additional alternative send time that has not yet been evaluated. If so, the method 400 returns to block 410 to select another alternative. If not, the method 400 continues to block 425.

Although the illustrated example depicts an iterative process for conceptual clarity (processing each alternative send time in turn), in some aspects, the computing system can process some or all of the alternatives in parallel. For example, in at least one aspect, the model may be a neural network where each neuron of a given layer of the network (e.g., at or just before the output) generates an interaction probability for a given time, allowing the model to process all alternative times in parallel. By using operations such as argmax or softmax on the outputs of these neurons, the system can thereby identify the target time.

In some embodiments, in addition to or instead of determining whether there are additional time(s) to be evaluated, the computing system can evaluate other termination criteria. In at least one embodiment, the computing system can determine whether the highest interaction probability that has been generated for the user exceeds some defined threshold value. For example, the computing system may iterate through alternative times and identify the highest probability that has been generated so far. If this value meets or exceeds a threshold, the computing system may determine to bypass the evaluation of further alternatives, and return this time as the suggested target. This can significantly reduce computational expense of identifying an optimal time, enabling the computing system to use the machine learning model to evaluate only a subset of the alternatives. By refraining from further processing, the computing system can reduce the latency of generating a target time and reduce the computational resources (e.g., processor time, power consumption, and the like) needed to generate the target time, while maintaining high interaction probability.

At block 425, the computing system selects a target send time (also referred to as a predicted optimal time, a predicted ideal time, and the like, as discussed above), from among the alternatives, based on the generated interaction probability of each. For example, as discussed above, the computing system may identify the time that was used to generate the highest interaction probability. In some embodiments, if two or more alternatives resulted in an equally high probability, the computing system can use other metrics or rules to select among them. For example, in one such embodiment, the computing system may select the alternative time that is nearest in the future (e.g., selecting a time that is one hour away rather than a time that is four hours away) if the interaction probabilities are the same (or are within a defined threshold distance from each other). In some embodiments, as discussed above, the computing system may determine whether the highest probability time has already occurred, and determine whether to wait for the optimal time on the subsequent day or to proceed with a lower-scored alternative time that has not-yet passed.

At block 430, the computing system facilitates communication at the selected send time. For example, if the computing system is a communication system (such as communication system 110 of FIGS. 1 and 2), the system can itself transmit, send, or otherwise provide the content to the user at the selected time. If the computing system is a machine learning system (such as machine learning system 115 of FIGS. 1 and 2), the system may indicate or otherwise provide the target send time to the communication system or another system, instructing or suggesting that the communication system send the content at the target time.

In some embodiments, prior to facilitating the communication, the computing system can determine whether the target send time satisfies one or more criteria, such as a minimum interaction probability. If not, the computing system may take other steps, such as generating or suggesting additional or alternative content that may be more engaging for the user. For example, the computing system may determine that the originally-planned content or method of communication is insufficient for the particular user (because they are unlikely to open or engage with it at all), and suggest or select alternative content (e.g., more engaging or interactive content), an alternative method of communication (e.g., text message rather than email), or make other changes to the communication in order to facilitate the interaction.

The method 400 can generally be performed for any number and variety of users, allowing the computing system to select ideal send times for each specific user based on their specific characteristics at this specific time. As the user characteristics change, the optimal transmission time may similarly shift. By using trained machine learning models, the computing system is able to dynamically drive communications in a way that maximizes the user engagement and experience.

### Example Method for Refining Content Transmission Machine Learning Models

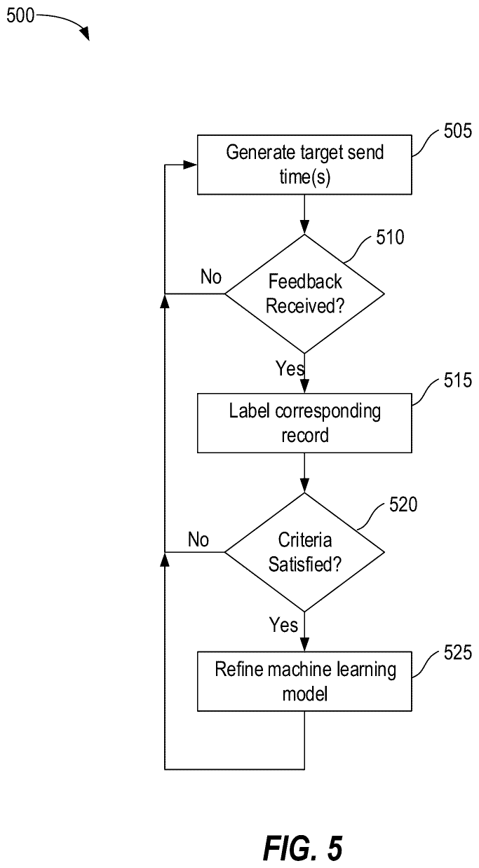

FIG. 5 is a flow diagram depicting an example method 500 for refining content transmission machine learning models. In some embodiments, the method 500 is performed by a machine learning system, such as machine learning system 115 of FIGS. 1 and 2. In other embodiments, the method 500 is performed by a communication system, such as communication system 110 of FIGS. 1 and 2.

At block 505, the computing system generates one or more target send times (e.g., selected, suggested, or predicted optimal times) for an intended recipient, as discussed above. For example, the computing system may use the method 400 of FIG. 4 to evaluate alternative times and select the optimal delivery time, then use the target time to facilitate communication with the recipient. In some embodiments, the computing system can additionally create a record for the communication or notification, where the record can include information such as identifying the intended recipient, indicating the specific user characteristics, indicating the time the communication was actually transmitted, and the like.

At block 510, the computing system determines whether any feedback has been received with respect to the communication. In some embodiments, this feedback includes an explicit indication or reply from the recipient user. For example, the user may indicate that the timing of the communication was good, acceptable, or poor. In some embodiments, the feedback corresponds to a determination as to whether the user opened the communication within the defined maximum time, as discussed above. In embodiments, the feedback can generally correspond to any desirable triggering criteria, as discussed above, such as the recipient user opening the content, interacting with the content, responding to the communication, and the like. In some embodiments, the feedback can correspond to information from a related device or system, such as a CPAP machine (e.g., whether the user started using the device more consistently), as discussed above.

If no feedback has been received, the method 500 returns to block 505 to continue generating target times for user(s). If feedback has been received, the method 500 continues to block 515. Although not included in the illustrated example, in some embodiments, the computing system may additionally or alternatively consider other criteria as well at block 510. For example, in one such embodiment, the computing system may determine whether the defined maximum duration of time (e.g., six hours) has passed. If the time has passed and the user has not opened or otherwise interacted with the communication, the computing system may use this determination as feedback (e.g., indicating that the target time did not successfully result in the user opening the content within the desired duration).

At block 515, the computing system labels the corresponding communication or notification record based on the feedback (or absence thereof). For example, as discussed above, the label may indicate the ground-truth(s) used in training, such as the time that elapsed between delivery or transmission of the notification and the corresponding user action (e.g., opening, responding, and the like). In some embodiments, the label is a binary value indicating whether the desired criteria are satisfied, such as whether the user opened the communication within a maximum duration, whether the user responded within the defined duration, and the like.

At block 520, the computing system determines whether one or more retraining or refinement criteria are satisfied. The retraining criteria may include a wide variety of considerations, such as a minimum number of labeled exemplars that should be available before retraining is performed, defined times when retraining should be performed (e.g., overnight), defined intervals between retraining (e.g., indicating that the model should be refined weekly), a minimum number of new exemplars where the user did not open or interact with the content within the window (e.g., indicating that the model predictions may be becoming less accurate), and the like.

If the criteria are not satisfied, the method 500 returns to block 505 and the computing system continues to generate target times and/or collect feedback for future training or refinement. If the criteria are satisfied, the method 500 continues to block 525, where the computing system refines the machine learning model based on the labeled records (labeled at block 515 based on the newly-received feedback). The method 500 then returns to block 505.

In this way, the computing system can generate updated models that can continue to generate accurate and reliable predictions even if conditions or realities change. In embodiments, continuous online updating (e.g., updating the model each time feedback is available) and/or periodic updating can thereby significantly improve the operations of the system and the usefulness and accuracy of the models.

### Example Method for Training Machine Learning Models to Predict Interaction Probability

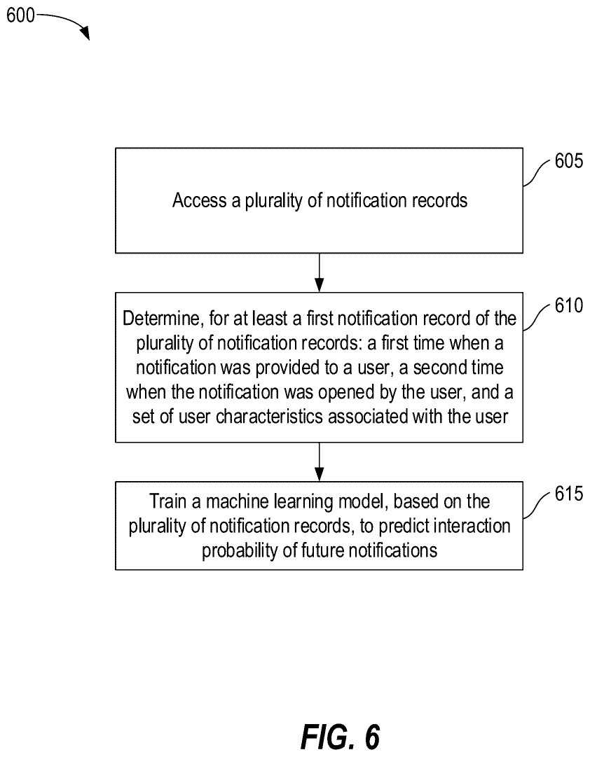

FIG. 6 is a flow diagram depicting an example method 600 for training a machine learning model to predict the probability of user interaction with future notifications. In some embodiments, the method 600 is performed by a machine learning system, such as machine learning system 115 of FIGS. 1 and 2. In other embodiments, the method 600 is performed by a communication system, such as communication system 110 of FIGS. 1 and 2.

At block 605, a plurality of notification records is accessed.

At block 610, for at least a first notification record of the plurality of notification records, a first time when a notification was provided to a user, a second time when the notification was opened by the user, and a set of user characteristics associated with the user are determined.

At block 615, a machine learning model is trained, based on the plurality of notification records, to predict interaction probability of future notifications.

### Example Method for Using Machine Learning to Transmit Notifications at Optimal Times

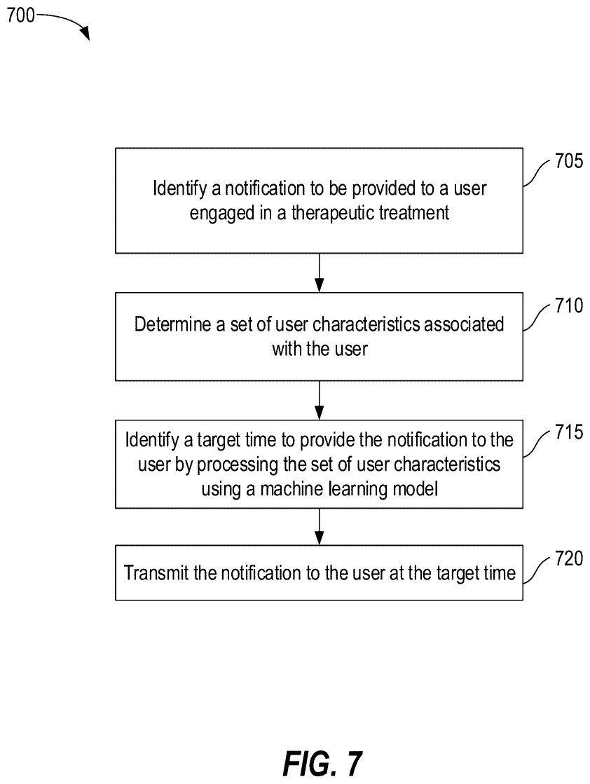

FIG. 7 is a flow diagram depicting an example method 700 for using a machine learning model to transmit notifications at an optimal time. In some embodiments, the method 700 is performed by a machine learning system, such as machine learning system 115 of FIGS. 1 and 2. In other embodiments, the method 700 is performed by a communication system, such as communication system 110 of FIGS. 1 and 2.

At block 705, a notification to be provided to a user engaged in a therapeutic treatment is identified.

At block 710, a set of user characteristics associated with the user is determined.

At block 715, a target time to provide the notification to the user is identified by processing the set of user characteristics using a machine learning model.

At block 720, the notification is transmitted to the user at the target time.

### Example Processing System for Improved Machine Learning

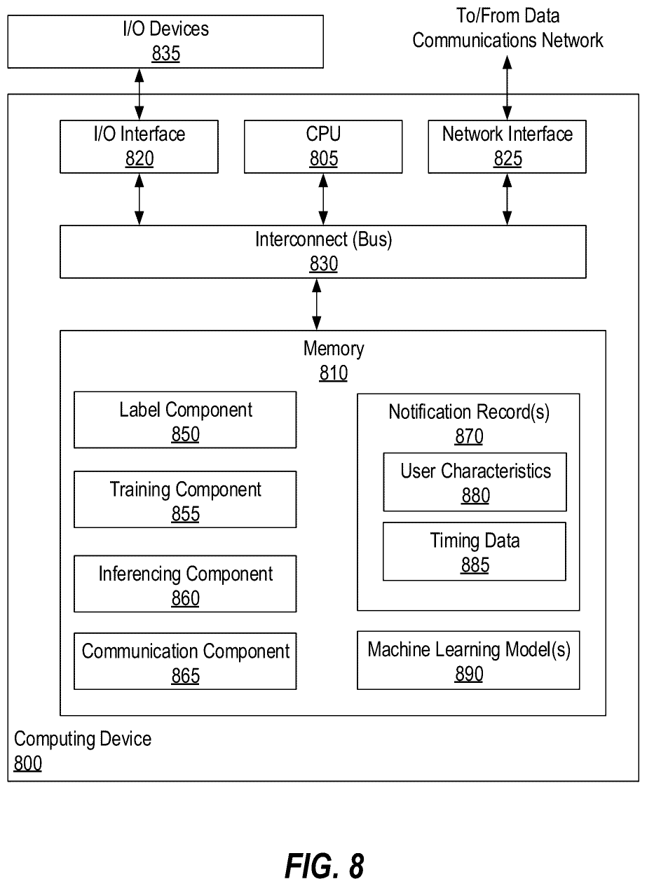

FIG. 8 depicts an example computing device 800 configured to perform various aspects of the present disclosure. Although depicted as a physical device, in embodiments, the computing device 800 may be implemented using virtual device(s), and/or across a number of devices (e.g., in a cloud environment). In one embodiment, the computing device 800 corresponds to the communication system 110 of FIG. 1 and/or FIG. 2, and/or the machine learning system 115 of FIG. 1 and/or FIG. 2.

As illustrated, the computing device 800 includes a CPU 805, memory 810, a network interface 825, and one or more I/O interfaces 820. In the illustrated embodiment, the CPU 805 retrieves and executes programming instructions stored in memory 810, as well as stores and retrieves application data (e.g., residing in a storage). The CPU 805 is generally representative of a single CPU and/or GPU, multiple CPUs and/or GPUs, a single CPU and/or GPU having multiple processing cores, and the like. The memory 810 is generally included to be representative of a random access memory. Although not included in the illustrated example, a storage of the computing device 800, if present, may include any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 835 (such as keyboards, monitors, etc.) are connected via the I/O interface(s) 820. Further, via the network interface 825, the computing device 800 can be communicatively coupled with one or more other devices and components (e.g., via a network, which may include the Internet, local network(s), and the like). As illustrated, the CPU 805, memory 810, network interface(s) 825, and I/O interface(s) 820 are communicatively coupled by one or more buses 830.

In the illustrated embodiment, the memory 810 includes a label component 850, a training component 855, an inferencing component 860, and a communication component 865, which may perform one or more embodiments discussed above. Although depicted as discrete components for conceptual clarity, in embodiments, the operations of the depicted components (and others not illustrated) may be combined or distributed across any number of components and systems. For example, a first system may be used to train the model(s) while a second system uses trained models for inferencing. For example, the depicted components could each be implemented as one or more cloud-based microservices tied together by application programming interfaces (APIs). Further, although depicted as software residing in memory 810, in embodiments, the operations of the depicted components (and others not illustrated) may be implemented using hardware, software, or a combination of hardware and software.

In one embodiment, the label component 850 is used to generate and/or label records (e.g., notification or communication records) based on how the corresponding recipient interacted with or responded to the notification (e.g., whether they opened it within a specified time period) in order to train and/or refine machine learning models, as discussed above. The training component 855 may generally be used to train and refine the models based on the labeled data. The Inferencing component 860 may be configured to use the trained model(s) to generate or identify optimal target times to transmit content, as discussed above.

In the illustrated example, the memory 810 further includes notification records 870 and one or more machine learning models 890. As discussed above, each notification record 870 may generally correspond to a respective communication or notification to a user. As illustrated, the notification records 870 each include or are associated with one or more user characteristics (e.g., indicating the attributes of the recipient user) and timing data 885 (e.g., indicating when the content was transmitted or provided, and when one or more user actions or interactions occurred, if any did occur). The notification records 870 may be generated (e.g., by the label component 850) and used (e.g., by the training component 855) to train the machine learning model(s) 890, as discussed above. Although depicted as residing in memory 810, the notification records 870 and machine learning model 890 may be stored in any suitable location, including storage, and may be stored in one or more other systems.

### Example Clauses

Implementation examples are described in the following numbered clauses:

Clause 1: A method, comprising: accessing a plurality of notification records; determining, for at least a first notification record of the plurality of notification records: a first time when a notification was provided to a user; a second time when the notification was opened by the user, and a set of user characteristics associated with the user; and training a machine learning model, based on the plurality of notification records, to predict interaction probability of future notifications.

Clause 2: The method of Clause 1, wherein training the machine learning model further comprises determining whether the second time is within a defined maximum length of time from the first time.

Clause 3: The method of any one of Clauses 1-2, wherein training the machine learning model to predict interaction probability comprises training the machine learning model to predict whether a future notification will be opened within the defined maximum length of time based at least in part on a time when the future notification will be provided.

Clause 4: The method of any one of Clauses 1-3, wherein the time when the future notification is provided corresponds to an hour of day.

Clause 5: The method of any one of Clauses 1-4, wherein the plurality of notification records correspond to communications related to ongoing therapeutic treatments.

Clause 6: The method of any one of Clauses 1-5, wherein the ongoing therapeutic treatments comprise treatment with at least one of (i) a continuous positive airway pressure (CPAP) device, (ii) a bilevel positive airway pressure (BiPAP) device, or (iii) an automatic positive airway pressure (APAP) device.

Clause 7: The method of any one of Clauses 1-6, wherein the communications comprise coaching content to improve the ongoing therapeutic treatments.

Clause 8: The method of any one of Clauses 1-7, wherein the set of user characteristics comprise at least one of: an age of the user, an average length of time the user used a medical device over a defined window of time, a standard deviation of the average length of time the user used the medical device over the defined window of time, a number of days that the user has used the medical device, a number of hours that the user used the medical device per day, an apnea index of the user, a sleep test type used by the user, or information relating to handset platforms the user uses.

Clause 9: A method, comprising: identifying a notification to be provided to a user engaged in a therapeutic treatment; determining a set of user characteristics associated with the user; identifying a target time to provide the notification to the user by processing the set of user characteristics using a machine learning model; and transmitting the notification to the user at the target time.

Clause 10: The method of Clause 9, wherein the notification corresponds to communications related to ongoing therapeutic treatments.

Clause 11: The method of any one of Clauses 9-10, wherein the ongoing therapeutic treatments comprise treatment with at least one of (i) a continuous positive airway pressure (CPAP) device, (ii) a bilevel positive airway pressure (BiPAP) device, or (iii) an automatic positive airway pressure (APAP) device.

Clause 12: The method of any one of Clauses 9-11, wherein the communications comprise coaching content to improve the ongoing therapeutic treatments.

Clause 13: The method of any one of Clauses 9-12, wherein identifying the target time comprises, for each respective alternative time of a plurality of alternative times, generating a respective probability that the user will open the notification within a defined maximum length of time if it is sent at the respective alternative time.

Clause 14: The method of any one of Clauses 9-13, wherein identifying the target time further comprises selecting an alternative time, of the plurality of alternative times, having a highest probability.

Clause 15: The method of any one of Clauses 9-14, wherein the set of user characteristics comprise at least one of: an age of the user, an average length of time the user used a medical device over a defined window of time, a standard deviation of the average length of time the user used the medical device over the defined window of time, a number of days that the user has used the medical device, a number of hours that the user used the medical device per day, an apnea index of the user, a sleep test type used by the user, or information relating to handset platforms the user uses.

Clause 16: The method of any one of Clauses 9-15, further comprising: determining whether the user opened the notification within a defined maximum length of time from the identified target time; and refining the machine learning model based on the determination.

Clause 17: A system, comprising: a memory comprising computer-executable instructions; and one or more processors configured to execute the computer-executable instructions and cause the processing system to perform a method in accordance with any one of Clauses 1-16.

Clause 18: A system, comprising means for performing a method in accordance with any one of Clauses 1-16.

Clause 19: A non-transitory computer-readable medium comprising computer-executable instructions that, when executed by one or more processors of a processing system, cause the processing system to perform a method in accordance with any one of Clauses 1-16.

Clause 20: A computer program product embodied on a computer-readable storage medium comprising code for performing a method in accordance with any one of Clauses 1-16.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access applications or systems (e.g., the machine learning system 115) or related data available in the cloud. For example, the machine learning system 115 could execute on a computing system in the cloud and train and/or use machine learning models. In such a case, the machine learning system 115 could train models to generate interaction probabilities, and store the models at a storage location in the cloud. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method, comprising:
accessing a plurality of notification records;
determining, for at least a first notification record of the plurality of notification records:
a first time when a notification was provided to a user;
a second time when the notification was opened by the user, and
a set of user characteristics associated with the user; and
training a machine learning model, based on the plurality of notification records, to predict interaction probability of future notifications.

2. The method of claim 1, wherein training the machine learning model further comprises determining whether the second time is within a defined maximum length of time from the first time.

3. The method of claim 2, wherein training the machine learning model to predict interaction probability comprises training the machine learning model to predict whether a future notification will be opened within the defined maximum length of time based at least in part on a time when the future notification will be provided.

4. The method of claim 3, wherein the time when the future notification is provided corresponds to an hour of day.

5. The method of claim 1, wherein the plurality of notification records correspond to communications related to ongoing therapeutic treatments.

6. The method of claim 5, wherein the ongoing therapeutic treatments comprise treatment with at least one of (i) a continuous positive airway pressure (CPAP) device, (ii) a bilevel positive airway pressure (BiPAP) device, or (iii) an automatic positive airway pressure (APAP) device.

7. The method of claim 5, wherein the communications comprise coaching content to improve the ongoing therapeutic treatments.

8. The method of claim 1, wherein the set of user characteristics comprise:
an age of the user,
an average length of time the user used a medical device over a defined window of time,
a standard deviation of the average length of time the user used the medical device over the defined window of time,
a number of days that the user has used the medical device,
a number of hours that the user used the medical device per day,
an apnea index of the user,
a sleep test type used by the user, or
information relating to handset platforms the user uses.

9. A method, comprising:
identifying a notification to be provided to a positive airway pressure device user engaged in a therapeutic treatment;
determining a set of user characteristics associated with the user;
identifying a target time to provide the notification to the user by processing the set of user characteristics using a machine learning model; and
transmitting the notification to the user at the target time.

10. The method of claim 9, wherein the notification corresponds to communications related to ongoing therapeutic treatments.

11. The method of claim 10, wherein the ongoing therapeutic treatments comprise treatment with at least one of (i) a continuous positive airway pressure (CPAP) device, (ii) a bilevel positive airway pressure (BiPAP) device, or (iii) an automatic positive airway pressure (APAP) device.

12. The method of claim 10, wherein the communications comprise coaching content to improve the ongoing therapeutic treatments.

13. The method of claim 9, wherein identifying the target time comprises, for each respective alternative time of a plurality of alternative times, generating a respective probability that the user will open the notification within a defined maximum length of time if it is sent at the respective alternative time.

14. The method of claim 13, wherein identifying the target time further comprises selecting an alternative time, of the plurality of alternative times, having a highest probability.

15. The method of claim 9, wherein the set of user characteristics comprise at least one of:
an age of the user,
an average length of time the user used a medical device over a defined window of time,
a standard deviation of the average length of time the user used the medical device over the defined window of time,
a number of days that the user has used the medical device,
a number of hours that the user used the medical device per day,
an apnea index of the user, and
a sleep test type used by the user, or
information relating to handset platforms the user uses.

9. method of claim 9, further comprising:
determining whether the user opened the notification within a defined maximum length of time from the identified target time; and
refining the machine learning model based on the determination.

17. A processing system, comprising:
a memory comprising computer-executable instructions; and
one or more processors configured to execute the computer-executable instructions and cause the processing system to perform an operation comprising:
identifying a notification to be provided to a user engaged in a therapeutic treatment;
determining a set of user characteristics associated with the user;
identifying a target time to provide the notification to the user by processing the set of user characteristics using a machine learning model; and
transmitting the notification to the user at the target time.

18. The processing system of claim 17, wherein identifying the target time comprises, for each respective alternative time of a plurality of alternative times, generating a respective probability that the user will open the notification within a defined maximum length of time if it is sent at the respective alternative time.

19. The processing system of claim 18, wherein identifying the target time further comprises selecting an alternative time, of the plurality of alternative times, having a highest probability.

20. The processing system of claim 17, further comprising:
determining whether the user opened the notification within a defined maximum length of time from the identified target time; and
refining the machine learning model based on the determination.
